(19) United States
(12) Patent Application Publication
(10) Pub. No.: US 2021/0109121 A1
(43) Pub. Date: Apr. 15, 2021

(54) AUTOMATIC TRANSFER BETWEEN TISSUE PROCESSING MODULES

(71) Applicant: Sakura Finetek Europe B.V.

(72) Inventors: Andries Dragt, Groet (NH) (NL); Robert Peter Jodies, Beusichem (NL)

(73) Assignee: Sakura Finetek Europe B.V., Alphen aan den Rijn (SH) (NL)

(21) Appl. No.: 16/600,263

(22) Filed: Oct. 11, 2019

(51) Int. Cl. G01N 35/00; G01N 1/31; A61B 6/00; G01N 1/36

(52) U.S. Cl. CPC G01N 35/0099; G01N 1/31; A61B 2034/2046; G01N 1/36; A61B 6/4458

(57) ABSTRACT

A method including engaging a carrier with a robot arm; and transferring the carrier by the robot arm from a first location outside a pathology instrument to a second location in the pathology instrument. A pathology assembly including a first pathology module; a second pathology module; and a robot arm operable to transfer a carrier operable to contain a plurality of tissue cassettes between the first pathology module and the second pathology module. A pathology assembly includes a processor coupled to the robot arm and comprising non-transitory machine readable instructions that when executed cause the robot arm to perform a method including engaging a carrier with the robot arm; and transferring the carrier by the robot arm from a first location outside a pathology instrument to a second location in the pathology instrument.

### FIELD

Tissue processing and more particularly automated loading and unloading of pathology modules or instruments.

### BACKGROUND

In pathology and histopathology laboratory environments, tissue taken from a body (e.g., a human body) is often placed in a cassette at a grossing station and then processed to remove and replace water. The tissue is then embedded in a paraffin (wax) block. The paraffin block is subsequently sliced in a microtome into thin sections. The thin sections are placed on slides that are stained and then examined.

Currently, many of the various processes from grossing to slide preparation and examination are performed at or in modular pathology instruments or stations. The individual modules may not be connected to each other and tissue samples in any form or shape are moved from module to module manually. For example, once a tissue sample is placed in a cassette at a grossing station, that cassette may be placed in a magazine that is operable to hold several cassettes (e.g., 20 cassettes, etc.). The magazine is then manually transferred by a technician from the grossing station to a tissue processor such as a Tissue-Tek Xpress® commercially available from Sakura Finetek U.S.A., Inc. of Torrance, Calif. and Tissue-Tek Xpress® is a trademark of Sakura Finetek U.S.A., Inc. In the tissue processing module, a tissue sample in each cassette in a magazine is processed to remove and replace water with paraffin. When the tissue processing is complete, a technician manually retrieves the magazine from the tissue processor and transfers the magazine to an embedding instrument such as a Tissue-Tek AutoTEC® also commercially available from Sakura Finetek U.S.A., Inc. and Tissue-Tek AutoTEC® is a trademark of Sakura Finetek U.S.A., Inc. In the Tissue-Tek AutoTEC®, the cassettes are individually removed from the magazine and subjected to an embedding process. Once the embedding process is complete, the cassettes are placed in slots in a door of the Tissue-Tek AutoTEC®. The door may then be manually removed by a technician when the embedding process for all the cassettes in the magazine is complete. The embedded cassettes may then be transferred manually by a technician to a microtomy station where tissue slides are prepared. The tissue slides are then manually placed into a slide stainer where the tissue on the slides are stained, after which they are moved to a coverslipper manually or automatically, after which the slides are then placed into a scanning device. Slides may then be manually placed into a folder or tray for examination by a pathologist.

### BRIEF DESCRIPTION OF THE DRAWINGS

FIG. 1 shows a top view of a portion of a pathology or histology laboratory including two pathology modules and a robotic transfer assembly associated with (free-standing or connected to) a bench and indicates an accessible region in which the robotic transfer assembly can function.

FIG. 2 shows a top side perspective view of the portion of a pathology or histology laboratory of FIG. 1 and shows a robot arm of the robotic assembly opening an input door of a first pathology module in the pathology or histology laboratory.

FIG. 3A shows a top side perspective view of a cassette magazine with cassettes loaded therein.

FIG. 3B shows the cassette magazine of FIG. 3A following the depression of a retainer onto the stack of cassettes loaded in the magazine.

FIG. 3C shows the cassette magazine of FIG. 3B and the insertion of a lid onto the front of the magazine.

FIG. 3D shows a front side view of a magazine assembly of two magazines connected by a handle.

FIG. 4 shows a top first side perspective view of the bench of FIG. 1.

FIG. 5 shows a top second side perspective of the bench of FIG. 1.

FIG. 6 shows a top first side perspective view of the bench or stand of FIG. 1 and shows an arm of the robot assembly placing a magazine in a compartment of a loading retort for drainage.

FIG. 7 shows a top first side perspective view of the bench of FIG. 1 and shows an arm of the robot assembly exposing cassettes in a magazine after placing the magazine in a cassette identification reader/capturer.

FIG. 8 shows a top first side perspective view of the bench of FIG. 1 and shows an arm of the robot assembly placing a handle on a pair of adjacent magazines in a compartment of the loading retort.

FIG. 9 shows the robot arm of the robotic assembly of FIG. 1 loading a magazine assembly into an input retort within a first pathology module in the pathology or histology laboratory.

FIG. 10 shows a top side perspective view of a robot arm of the robotic assembly of FIG. 1 removing a magazine assembly from an output retort within a first pathology module in the pathology or histology laboratory.

FIG. 11 shows a top first side perspective view of the bench of FIG. 1 and shows a magazine assembly after being removed from a first pathology module and placed in a magazine storage area and shows the robotic assembly removing a handle from each of the magazines of the magazine assembly.

FIG. 12 shows a top side perspective view of a portion of a second pathology module in the pathology or histology laboratory of FIG. 1 and shows a robot arm of the robotic assembly opening an input door of the second pathology module in the pathology or histology laboratory.

FIG. 13 shows a top side perspective of the portion of the second pathology module shown in FIG. 12 and shows a robot arm of the robotic assembly loading a magazine into a slot in the input door of the second pathology module.

FIG. 14 shows a top side perspective of the portion of the second pathology module shown in FIG. 12 and shows a robot arm of the robotic assembly closing the input door.

FIG. 15 is a flow chart of a method of handling a carrier including loading and unloading of a carrier into pathology modules in a pathology or histology laboratory.

FIG. 16 shows a top view of a portion of a pathology or histology laboratory including four pathology modules and two robotic transfer assemblies associated with (free-standing or connected to) a bench and indicates an accessible region in which each robotic transfer assembly can function.

### DETAILED DESCRIPTION

An assembly or system for processing tissue samples in a pathology or histology laboratory environment is disclosed. A pathology assembly may include a first pathology module; a second pathology module; and at least one robot arm operable to transfer a carrier operable to contain a plurality of tissue cassettes or slides between the first pathology module and the second pathology module. The first pathology module in the pathology assembly may include a grossing station. The second pathology module may include a tissue processing instrument. A pathology assembly may also include more than two pathology modules and the robot arm is operable to transfer a carrier between the modules. For example, a first pathology module may include a grossing station, a second pathology module may include a tissue processing instrument, a third pathology module may include an embedding instrument, a fourth module may include a sectioning and slide preparation instrument, a fifth module may include a slide staining instrument, a sixth module may include a slide coverslipping instrument and a seventh module may include a slide scanning instrument. A robot arm may be operable to transfer the carrier between the modules, such as from the first pathology module to the second pathology module and from second pathology module to the third pathology module and so forth. A robot arm may not be limited to transferring a single type of carrier. For example, tissue cassettes may be introduced into a tissue processing instrument such as the Tissue-Tek AutoTEC® in a magazine and removed from that instrument in a door. A robot arm may be operable to transfer either a magazine or a door or other carrier (e.g., basket that may contain several slides) with one or multiple end effectors. In another example, a carrier could be single slide or a single tissue cassette.

In addition to the pathology modules and at least one robot arm to transfer a carrier to or between pathology modules, a pathology assembly may include a reader or capturer operable to read or capture an identifier on the carrier. A pathology assembly may also include both a reader and a capturer. Examples of identifiers include a bar code, a radio frequency identification (RFID), text or color. Suitable readers include bar code readers, RFID readers, dot matrix code (DMC) or cameras using vision-based identification methods. A suitable capturer includes a digital imager or camera. The information from the carrier that is read and/or captured may be saved (e.g., saved in a computer memory) and used to track the carrier through the pathology laboratory.

A pathology assembly is also disclosed that may include a cart, bench or stand, rail or gauntry (e.g., a ceiling or wall-mounted gauntry) including at least one robot arm connected to a base and a processor (robot controller) coupled to the robot arm and including non-transitory machine readable instructions that when executed cause the robot arm to perform a method including engaging a carrier with the robot arm and transferring the carrier by the robot arm from a first location outside a pathology instrument to a second location in the pathology instrument and/or transferring the carrier by the robot arm from the first pathology instrument to a second pathology instrument. The at least one robot arm may be mounted on a stationary work bench positioned in an area providing the at least one robot arm or an end effector thereof access to the pathology modules (e.g., an accessible region or area). The at least one robot arm may alternatively be moveable around an accessible region or area such as by being mounted on a cart with rollers or wheels and having a computer- or operator-controlled motor to move through the accessible region or area (e.g., to power the wheels and maneuver the cart). The work bench or cart may have a work surface having an area of a size to provide an area to prepare and store tissue cassettes, magazines, slides, etc. and/or to provide an area for instruments such as capturers and/or readers (e.g., a bar code reader) and labelers. The at least one robot arm may alternatively be a free-standing stationary or moveable unit (i.e., not connected to a work bench or cart) including, for example, a base or stand, optionally with wheels and a motor to drive and maneuver the wheels as a self-propelled unit, and a robot controller to control a movement of a robot arm and/or an end effector thereon and possibly a movement of the base or stand. The at least one robot arm may alternatively be connected to wall or ceiling of a pathology or histology laboratory, either in a stationary position (connected at its base to a single immoveable area) or moveable thereon (e.g., connected at its base to a rail on the wall or ceiling on which the at least one robot arm may move). The at least one robot arm may further alternatively be connected to a gantry either in a stationary position or moveable thereon, wherein the gantry includes a frame structure or support platform spanning a portion of a pathology or histology laboratory.

The pathology assembly may include a reader or capturer operable to read or capture an identifier on the carrier. As noted, such a reader or capturer may be mounted on a work bench or cart in an accessible region or area. The pathology assembly may also include storage areas for cassettes, magazines and/or slides as well other equipment (e.g., a cassette labeler) and such storage areas and/or equipment may be positioned on a work bench or cart if present.

A method for processing tissue samples in a pathology or histology laboratory environment is further disclosed. A method may include engaging a carrier with a robot arm and transferring the carrier by the robot arm from a first location outside a pathology instrument to a second location in the pathology instrument. Examples of a carrier may be a magazine, door, rack or basket that is operable to contain one or more tissue cassettes or slides, a cassette and a slide. A carrier such as a magazine or basket may include a handle and the robot arm may include an end effector so that transferring the carrier by the robot includes gripping the handle by the end effector. A pathology laboratory environment may include more than two pathology modules and the method for processing tissue samples may include transferring a carrier or carriers by a robot arm between the modules.

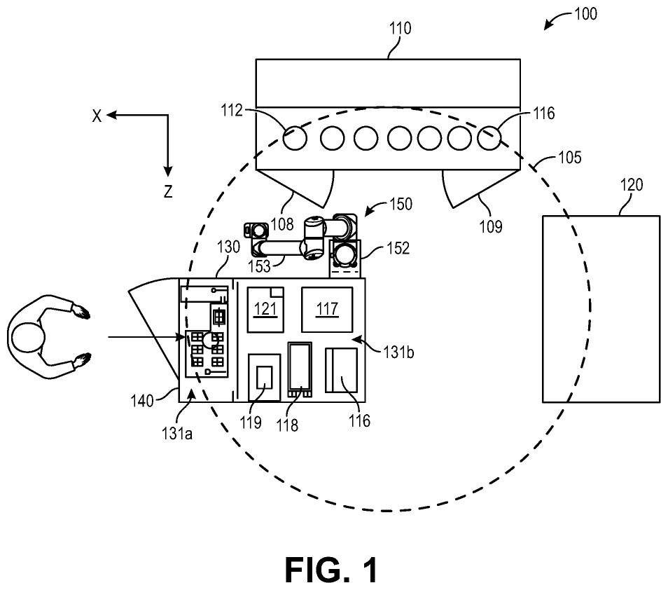

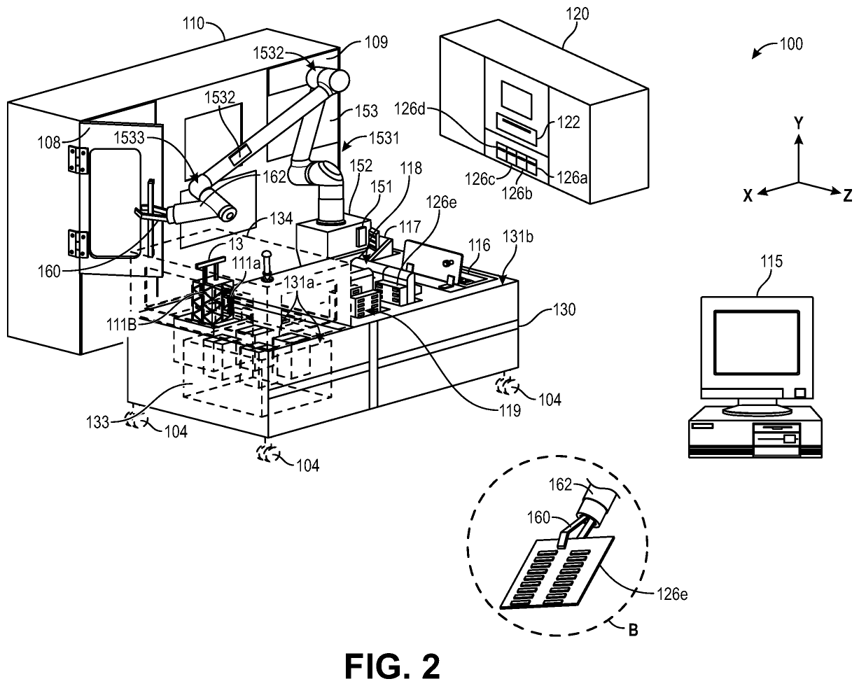

FIG. 1 shows a top view of a portion of a pathology or histology laboratory including two pathology modules and a robotic transfer assembly. FIG. 2 shows an oblique perspective view of a similar portion of the laboratory. In this example, portion 100 of the laboratory includes pathology module 110 that is, for example, a Tissue-Tek Xpress® and pathology module 120 that is, for example, a Tissue-Tek AutoTEC®, both commercially available from Sakura Finetek U.S.A., Inc. Each of pathology module 110 and pathology module 120 is a stand-alone instrument, meaning the modules are not physically connected and can be manually operated. The modules may be, for example, electronically connected through, for example, a communications link via controller or computer 115. In this example, pathology module 110 and pathology module 120 are arranged so that a front side of each module is facing bench 130. More specifically, in this example, pathology module 110 and pathology module 120 are arranged generally orthogonally relative to the other. Pathology module 110 includes a length dimension that extends in an x-direction and pathology module 120 includes a length dimension that extends in a z-direction. Other arrangements are possible, including, a linear or stacked arrangement of modules. Bench 130 is shown as a stationary bench. In another example, bench 130 may also be movable (a cart), such as a motorized bench on optional wheels or rollers 104 that is electrically movable through a portion of a pathology or histology laboratory. Movement of a movable bench 130 may be controlled by machine-readable instructions transmitted from computer 115.

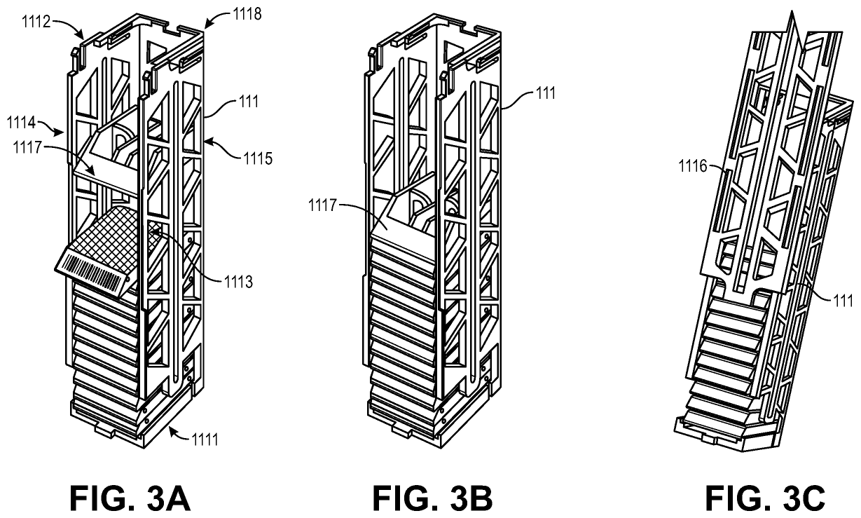

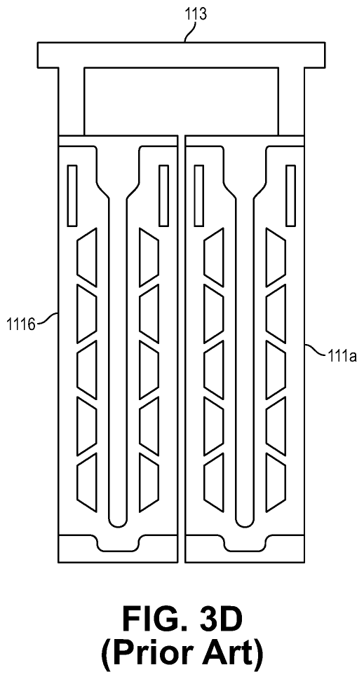

Pathology module 110 of a Tissue-Tek Xpress® accepts tissue samples in cassettes. Before a cassette goes into the Tissue-Tek Xpress®, the cassette is placed in a magazine. Representative magazines include Tissue-Tek AutoTEC® magazines and Tissue-Tek Xpress® magazines. Each magazine may hold as an example 20 cassettes in a stacked arrangement (one cassette stacked on top of the other). FIGS. 3A, 3B and 3C show steps in loading cassettes each containing a tissue sample into a magazine. As shown in FIG. 3A, magazine 111 is, for example, a molded plastic container having top 1112, bottom 1111, two opposing sidewalls 1113 and 1114 and rear wall 1115. The cassettes are loaded such that the front side wall faces the front of the magazine. Each cassette has a front side wall that may have identification information such as a machine readable or hand-written label on it containing patient and/or processing information. Once all the cassettes are placed in the interior of magazine 111, retainer 1117 may be pushed downward to secure the cassettes (FIG. 3B), then lid 1116 is slid downward onto the sidewall edges (sidewall 1113 and sidewall 1114) to form a front wall of the magazine (FIG. 3C). In one example, the label of each cassette may be read after placing the cassette in a magazine, but before placing lid 1116 on a front of the magazine. Representatively, the pathology laboratory may have a cassette reader/capturer such as described in U.S. patent application Ser. No. 16/153,634 titled “Tissue Cassette Reader” which is incorporated herein by reference. U.S. patent application Ser. No. 16/153,634 describes a stand-alone device that may, for example, be placed on bench 130 or in another area of the laboratory where tissue cassettes may be loaded into magazines. One or more magazines may be placed in the stand-alone device and then the device is operable to capture an image (e.g., a two-dimensional or three-dimensional image) of an identifier on a cassette, such as a bar code on a front side wall. A magazine may then be removed from the device and removable lid 1116 may be placed over the front of magazine 111 to prepare it for loading into a Tissue-Tek Xpress®.

Before loading into a Tissue-Tek Xpress®, two magazines are positioned side-by-side and a handle is attached to transport the two magazines together. As shown in FIG. 3A, top 1112 of magazine 111 has a lip onto which a handle base can slide (e.g., from a position behind the magazine toward the front). FIG. 3D shows magazine 111a and magazine 111b connected through removable handle 113 to form a magazine assembly. The magazine assembly of magazine 111a and magazine 111b with handle 113 attached are ready to go into pathology module 110 for tissue processing of the cassettes in each magazine. In one example, handle 113 may include a an identifier such as a bar code, RFID or any other. Prior to loading the magazine assembly into pathology module 110, the magazines in the magazine assembly may be submerged in a pre-processing fluid such as a fixative so that the tissue samples do not dry out.

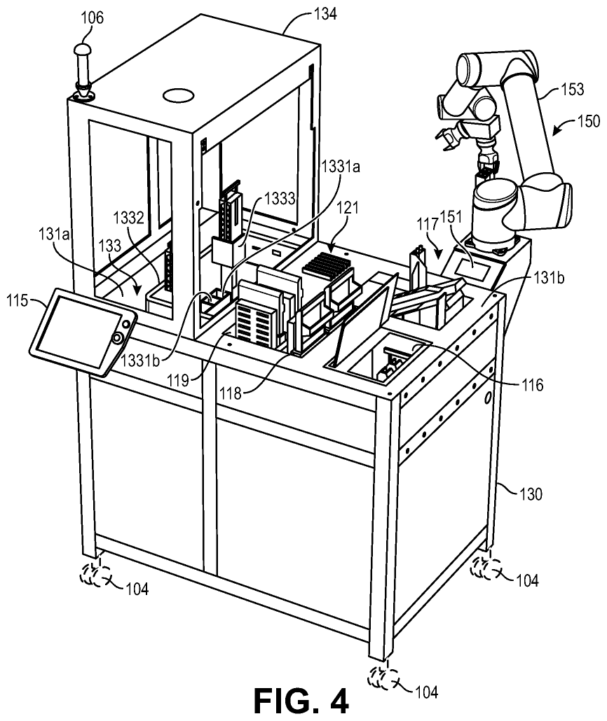

Returning to FIG. 1 and FIG. 2, bench 130 includes a superior work surface and legs or sidewalls extending to the floor. The work surface may be a horizontal surface that is 36 inches to 48 inches above the floor. The superior work surface of bench 130 includes surface portion 131a and surface portion 131b. Each of surface portion 131a and surface portion 131b may have an area of a size to provide an area to prepare and store tissue cassettes in preparation for processing or assemble magazines containing tissue cassettes for processing in pathology module 110 and/or pathology module 120 and an area for a tissue cassette reader to record identification information about each cassette. Each area may also provide an area for storage of magazines or output doors of processed cassettes or empty magazines or output doors. FIG. 4 shows a top side view of an example of bench 130. Computer 115, in this example, is connected to a side of bench 130. In this example, surface portion 131a includes retort 133 that may accommodate tissue cassette carriers such as cassette magazines that are to be transferred to pathology module 110. In this example, one or more magazine assemblies may be loaded into loading retort 133 that contains a pre-processing fluid such as a fixative. Overlying portion 131a in one example may be hood 134 that is operable to contain volatile fumes or gases in the retort. Disposed on a top portion of hood 134 is light pole 106 that includes one or more lights that serve to indicate when robot apparatus 150 is performing an action.

Surface portion 131b of bench 130 in this example includes magazine storage area 116 that is operable to contain magazines that have cassettes that have been processed, for example, in a Tissue-Tek Xpress tissue processing module. A volume of magazine storage area 116 may contain slots for containing individual magazines or pairs of magazines. Magazines may be contained in magazine storage area 116 individually or in pairs with or without a handle (handle 113, FIG. 3D). Pairs of magazines including a handle may be positioned in magazine storage area 116 and then the handle may be removed. Accordingly, magazine storage area 116 may provide a containment area or volume for magazines so that a handle connecting a pair of magazines may be removed robotically or by other means. Magazine storage area 116 may include an electric or Joule heater therein to keep processed cassettes warm until the cassettes are transferred into another pathology module (e.g., pathology module 120).

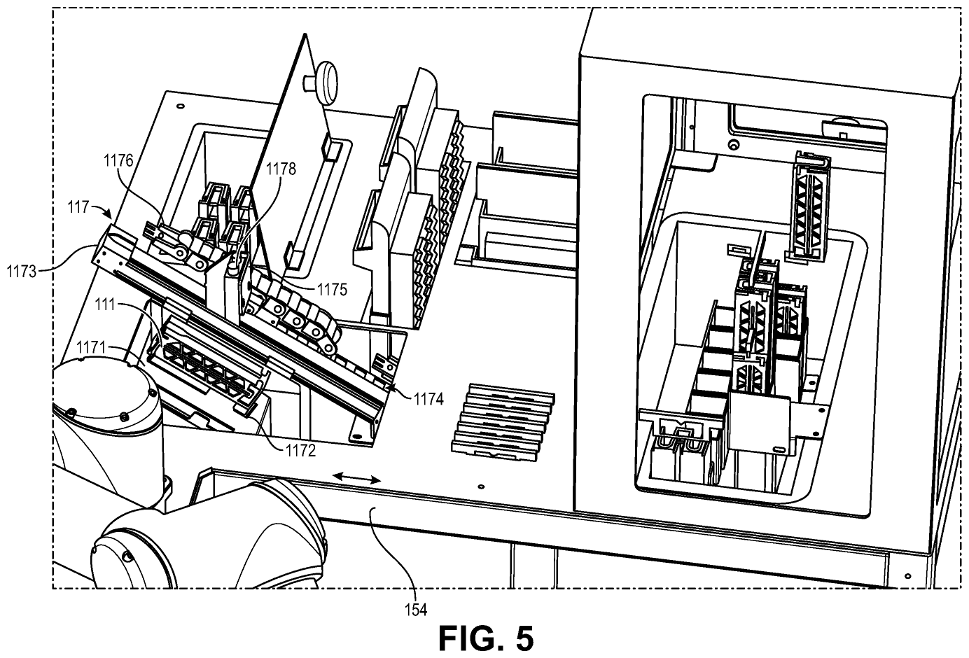

Surface portion 131b of bench 130 also includes cassette reader/capturer 117 in one area. Cassette reader/capturer 117 is operable to engage a magazine and read and/or capture an image of an identifier on a cassette in the magazine. FIG. 5 shows a magnified top side view of bench 130 opposite the side view of FIG. 4. In this example, cassette reader/capturer 117 includes dock 1171 that is set off at an angle relative to surface portion 131 of bench 130. Dock 1171 includes one or more tabs 1172 to support a position of a magazine disposed lengthwise in a loaded position on the dock. Representatively, dock 1171 projects from surface portion 131 at an angle of 30 degrees to 45 degrees. Cassette reader/capturer 117 also includes support arm 1173 also projecting as a cantilever from surface portion 131 at a similar angle as dock 1171. Support arm 1173 may have a shape of a rectangular prism with a length, L, on the order of 30 centimeters (cm) to 45 cm. Dock 1171 is connected to a side of support arm 1173 (a side opposite magazine storage area 116) at approximately a midpoint of support arm 1173. A superior surface of support arm 1173 may have track 1174 extending along its length. Connected to support arm 1173 and projecting perpendicularly from a superior surface thereof is bracket 1175. Bracket 1175 is operable to engage reader and/or camera 1178 (e.g., in one example both a bar code or RFID reader and a camera) at a position above or superior to dock 1172 (e.g., the reader and/or camera having a lens(es) facing dock 1172). Bracket 1175 may be an approximately u-shaped bracket having a tongue at its base extending in an inferior direction into track 1174 in support arm 1173. One arm of bracket 1175 projects laterally above dock 1171. Connected to the laterally projecting arm is reader and/or camera 1178. Bracket 1175 may be connected to track 1176 that is disposed adjacent support arm 1173 or within support arm 1173. Track 1176 is connected to a step motor. Track 1176 is operable to advance bracket 1175 (and reader and/or camera 1178 thereon) a distance equivalent to a thickness of a cassette. In this manner, when a magazine is positioned in dock 1171 with its lid removed to expose cassettes therein, reader and/or camera 1178 can read and/or image an identifier on a front sidewall of each cassette in the magazine. Since the front sidewall is often angled at approximately 30 degrees to 45 degrees relative to a base of the cassette, the projection of support arm at an opposite 30 to 45 degree angle relative to surface portion 131 allows, for example, a lens of reader and/or camera 1178 to be positioned generally parallel to a front sidewall of a cassette.

Surface portion 131b of bench 130 also includes storage area 118 operable for storage of, for example, doors containing cassettes that have been processed in a Tissue-Tek AutoTEC® and storage area 119 for storage of empty doors (doors without cassettes). Storage area 118 may include an electric or Joule heating element therein to heat cassettes in doors after embedding. FIG. 4 also shows storage area 121 for storage of magazine handles.

FIG. 1 and FIG. 2 also show robot apparatus or assembly 150 associated with bench 130. Robot apparatus or assembly 150 may be connected to surface portion 131b of bench 130 as illustrated or may be a free standing unit adjacent to bench 130 (as indicated by the dashed lines in FIG. 1). Robot apparatus 150 may be positioned (e.g., mounted to bench 130) approximately in a center of the area occupied by the portion of the laboratory including pathology module 110 and pathology module 120 so that it may be approximately equidistant from each module. Alternatively, robot apparatus 150 may be positioned (e.g., mounted to bench 130) further from one module than another module. In one example illustrated in FIG. 5, bench 130 may include track or rail 154 that extends a portion of a length on one side of the bench. Robot apparatus 150 may be mounted on track 154 and be operable to move along the track from one end of bench 130 toward another end. A free-standing robot apparatus may have wheels or rollers and a motor controlled, for example, by computer 115 (e.g., controlled by non-transitory, machine-readable instructions contained in computer 115 and transmitted to robot apparatus 150) allowing robot apparatus 150 to be moved, for example, from one end of bench 130 to the other. Robot apparatus 150 may have dimensions that allow robot apparatus 150 to grasp individual magazines, pairs of magazines connected by a single handle (a “magazine assembly”) from loading retort 133 and transfer such magazines or assemblies, such as one magazine or one magazine assembly at a time, into or out of loading retort 133, into or out of cassette reader/capturer 117, into or out of pathology module 110 and to transfer such assemblies from pathology module 110 to pathology module 120 (e.g., one magazine at a time).

Robot apparatus 150 includes robot controller 151 housed in or connected to base 152 of the robot apparatus (see FIG. 1). Robot controller 151 may control robot arm motion and processing tasks to be performed by robot apparatus 150, barcode scanner control and equipment interfacing. Robot controller 151 is linked either through hardwiring or wirelessly to computer 115. Machine readable program instructions are transmitted between computer 115 and robot controller 151 (e.g., from computer 115 to direct robot controller 151) to perform a desired protocol and, where robot apparatus 150 is a free-standing structure that includes a motor to move the structure, to maneuver robot apparatus. Robot controller 151 may transmit a signal or signals back to the computer 115 confirming the instructions and/or after completing an action directed by computer 115.

Robot apparatus 150 includes robot arm 153 mounted to base 152. Robot arm 153 may be operable to or configured to perform actions within accessible region 105, depicted in cross-hatching in FIG. 1. As illustrated, accessible region 105 encompasses portions of pathology module 110, pathology module 120 and bench 130, allowing an end of robot arm 153 to access the pathology modules and bench 130. Such actions may include transfer or transport of carriers between bench 130, pathology module 110 and/or pathology module 120. Robot arm 153 may be a multi-joint arm having, for example, three, four or six or more rotational joints (e.g., rotational joint 1531 (shoulder), rotational joint 1532 (elbow), rotational joint 1533 (wrist) and rotational joint 1534 (base) illustrated and possibly one or more translational joint). The rotational joints permit movement of robot arm 153 in arcuate paths about a horizontal plane or rotary action along a joint axis. A translational or prismatic joint permits movement of the robot arm 153 in an axial direction (along a joint axis). Robot arm 153 may be controlled by signals from robot controller 151 to position a distal end of the robot arm at a location within accessible region 105 (see FIG. 1). One example of a suitable robot apparatus 150 is a Universal Robots UR 10 or UR 5 Collaborative Robot, commercially available from Universal Robots, Denmark.

Robot apparatus 150 includes one or more end tools or end effectors 160 attached to a distal end of robot arm 153 for picking and placing pathology instruments. Representatively, each of the one or more end effectors 160 employ a parallel gripper, which is sized to accommodate the pathology instruments such as cassette carriers (e.g., magazines, module doors, baskets) as well as to open and close doors of pathology module 110 and pathology module 120. The one or more end effectors 160 are controlled by signals from robot controller 151 to, for example, automatically rotate the gripper(s), open the gripper(s), and/or dose the gripper(s). FIG. 1 shows robot apparatus 150 having one end effector. FIG. 4 shows robot apparatus 150 including two end effectors. The following paragraphs describe a robot apparatus having a single end effector. It is appreciated that the description of the system, the apparatus and its use is applicable to a robot apparatus having one or more end effectors.

Robot apparatus 150 may optionally include a tool changer to change the end effector or tool. FIG. 2 shows tool changer 162 that is connected between robot arm 153 and end effector 160 to permit removable attachment of end effector 160. Tool changer 162 may be any suitable tool changer that is compatible with the robot arm 153. The tool changer 162 may, for example, be an electrically-activated latch to attach end effector 160 to tool changer 162. A switch of tool changer 162 may be energized and de-energized by robot controller 151 to apply and remove, respectively, the source holding the end effector 160. Tool changer 162 facilitates automated changing of end effector 160 via an end effector nest located in accessible region 105.

Still referring to FIG. 2, to optional tool changer 162 on robot arm 153 reader and/or capturer 1532 that is operable to read and/or capture identification information on a carrier, such as an identifier (a label) on handle 113 or magazine 111a and 111b. In this manner, prior to and/or after transporting a carrier to or from a pathology module, an identifier associated with the carrier may be read or captured to track the progress or location of the tissue cassettes contained in the carrier.

Operation of robot apparatus 150 to perform actions within accessible region 105 may be controlled by and displayed to a user or operator on computer 115. Computer 115 may include a processor connected physically (e.g., through wiring) or remotely (e.g., wirelessly) to robot controller 151. Computer 115 may also include a display such as a touch screen display allowing a user to, for example, start and stop activity of robot apparatus 150.

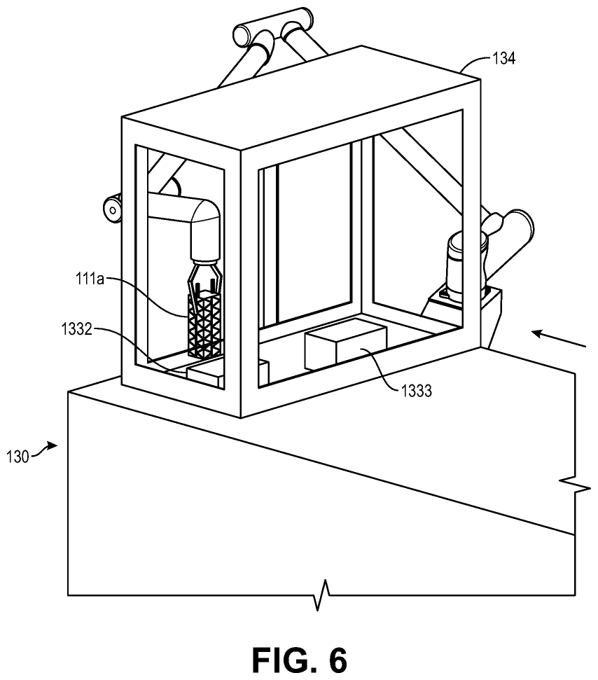

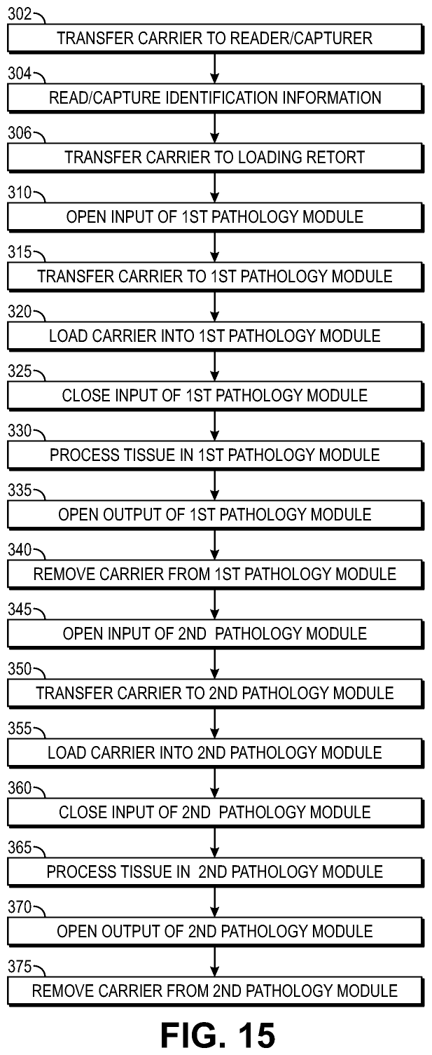

In one example, it is contemplated that the processing of tissue cassettes through pathology module 110 (e.g., a Tissue-Tek Xpress®) and pathology module 120 (e.g., a Tissue-Tek AutoTEC®) may be done without human intervention by using robot apparatus 150 to prepare, load and unload cassettes from each module. FIGS. 6-14 show representative actions of robot apparatus 150 to prepare, load and remove cassettes in and from pathology module 110 (a Tissue-Tek Xpress®) and pathology module 120 (a Tissue-Tek AutoTEC®). FIG. 15 is a flow chart for processing tissue cassettes through both pathology module 110 and pathology module 120. FIG. 4 and FIG. 5 show several magazines in loading retort 133. The magazines may include one or more tissue cassettes prepared by an operator for tissue processing in pathology module 110 (e.g., a Tissue-Tek Xpress®). An operator may manually load individual magazines into loading retort 133. Once a magazine is initially loaded in loading retort 133, physical human interaction (e.g., handling) with the magazine or cassettes therein may cease until, for example, a tissue sample in the cassette is embedded and removed from pathology module 120 or, in another example, until after a tissue section from the tissue sample is prepared, stained and even imaged, depending on the modules that a robot apparatus is programmed and engaged to interact.

Referring again to FIG. 4 and FIG. 5, volume of loading retort 133 may have individual compartments with each compartment having dimensions slightly greater than width and depth dimensions of a magazine so that a magazine may be accommodated in a compartment or slot. FIG. 4 representatively shows adjacent compartments 1331a and 1331b. In one example, identifiers on each of the tissue cassettes in a magazine have been read and/or captured through the use of a reader/capturer such as described in U.S. patent application Ser. No. 16/153,634 titled “Tissue Cassette Reader” and the processing protocol for each magazine confirmed at computer 115. For example, the reader/capturer may be electrically or wirelessly linked to computer 115 and is operable to transfer data (e.g., identifier information) to computer 115. Computer 115 may include a database or may be linked to a database such as a Laboratory Information System (LIS) directly or via a middleware that contains processing information for tissue samples that are to be processed in the laboratory. When computer 115 receives identifier information from a reader/capturer in the laboratory, non-transitory machine-readable processing instructions associated with computer 115 allow a comparison of the transmitted information to the database information.

When one or more magazines or magazine assemblies are positioned in loading retort 133, an operator may start the activity of robot apparatus 150 by, for example, initiating a start sequence at computer 115. Alternatively, loading retort 133 may include one or more sensors that indicate the presence of a magazine or magazine assemblies allowing a start sequence of robot apparatus 150 to begin automatically. For example, loading retort 133 may include one or more photo eyes where a light (e.g., a laser light) is directed across an upper area of the retort to a second sensor. When the path of the directed light is obstructed by the presence of, for example, a magazine or a magazine assembly, a signal can be received by computer 115 to indicate the presence of a magazine or magazine assembly and computer 115 can initiate a start sequence. Alternatively, robot apparatus 150 is operable to probe compartments in loading retort 133 one by one (e.g., compartment 1331a, compartment 1331b, etc.) for a magazine. For example, robot apparatus 150, through non-transitory, machine readable control sequences in robot controller 151 and possibly transmitted to robot controller 151 from computer 115, directs end effector 160 at a distal end of robot arm 153 to position itself in front of a door of hood 134. The door may be engaged by an actuator that is released by a signal from robot apparatus (e.g., a light signal sent from robot arm 153 to a sensor associated with the actuator). A release of the actuator may cause the door to open providing access from robot arm 153 and end effector 160 to enter hood 134 and compartment by compartment probe loading retort 133. The probing may be done by physical contact between end effector 160 and a top of a magazine or by a reflected light signal sent and received at robot arm 153 or end effector 160.

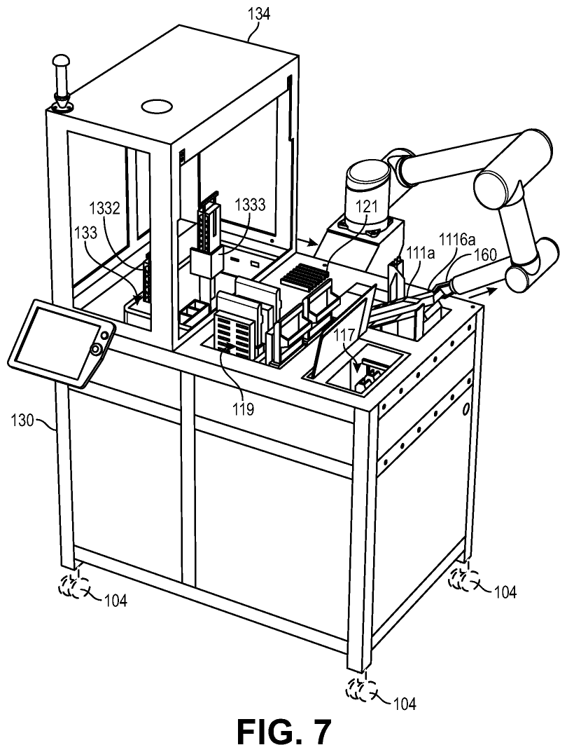

If a magazine is present in a compartment in loading retort 133, a start sequence may be initiated with or without direction from computer 115 (e.g., a start sequence may be initiated by program instructions associated with robot controller 151 or robot apparatus 150). In one example, robot controller 151 directs end effector 160 at a distal end of robot arm 153a to grasp the magazine and place it in drip tray 1332 positioned above loading retort 133 within hood 134. FIG. 6 shows a base of robot 150 moved on rail 154 from a position at one end of bench 130 near cassette reader/capturer 117 (e.g., see FIG. 5) to an end near loading retort 133 and shows end effector 160 positioned inside of hood 134 over loading retort 133 and grasping magazine 111a above drip tray 1332. Placing a magazine in drip tray 1332 allows carried or excess fluid (e.g., formalin) retained by the magazine from loading retort 133 to be drained into loading retort 133 and reduces spillage outside of the loading retort. After a programmed time in drip tray 1332 (e.g., 20 seconds to 60 seconds), end effector 160 will again grasp the magazine in drip tray 1332 or if end effector maintained its grasp for the programmed time and transfer it from the drip tray to cassette reader/capturer 117 (block 302, FIG. 15). At cassette reader/capturer 117, robot apparatus through end effector 160 loads the magazine into dock 1171 of cassette reader/capturer 117 with the lid of the magazine positioned upward and then may begin to remove the lid. FIG. 7 shows a base of robot apparatus 150 moved from a position near hood 134 to a position closer to cassette/reader capturer 117 and magazine 111a loaded in or engaged by cassette reader/capturer 117. FIG. 7 also shows end effector 160 grasping lid 1116a of magazine 111a and sliding (pulling) the lid away from the bottom of magazine 111a toward the top to expose cassettes in the magazine. Where magazine 111a is not completely filled from bottom to the top with cassettes or otherwise has space toward the top devoid of cassettes or positions for cassettes, end effector 160 need not separate lid 1116a from the sidewalls of magazine 111a. End effector 116 and robot apparatus only need to move lid 1116a a distance from bottom to top of the magazine to expose all the cassettes in the magazine.

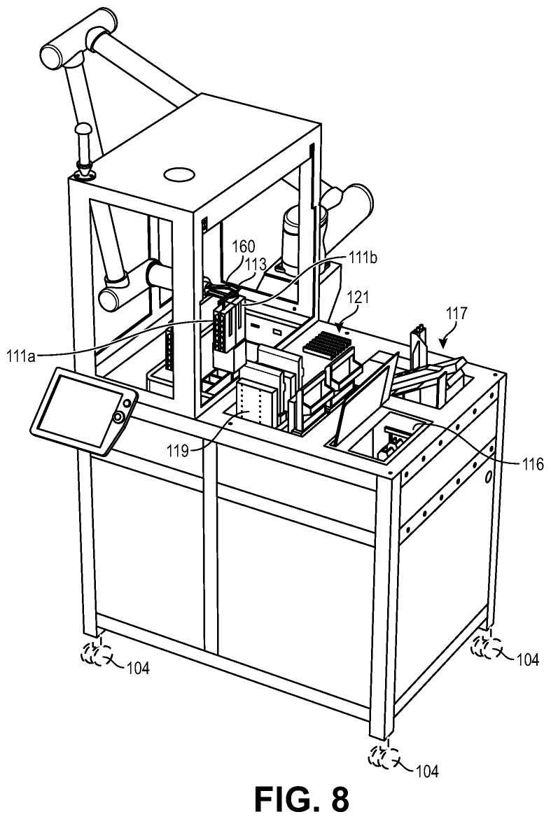

Once all the cassettes in a magazine are exposed while the magazine is positioned in cassette reader/capturer 117, reader and/or camera 1178 can begin scanning, reading and/or capturing (e.g., imaging) any identifier on a front sidewall surface the cassettes (block 304, FIG. 15). The scan, read and/or captured information may be provided to computer 115. Once the scanning, reading and/or capturing is complete, robot apparatus 150 through end effector 160 may reinstall lid 1116a on magazine 111a to secure all the cassettes in the magazine. The reinstallation of lid 1116a can involve grasping lid 1116a and reversing the previous sliding (pulling) motion that removed the lid. The magazine may be returned to loading retort 133 and placed in docking compartment 1333 therein submerged in a fluid (e.g., formalin) (block 306, FIG. 15). When a second magazine is available in loading retort 133, the second magazine will undergo a similar procedure as the first magazine (e.g., removed from loading retort 133; identifiers scanned, read and/or captured in cassette reader/capturer 117; returned to loading retort 133). The second magazine will be placed in docking compartment 1333 immediately adjacent the first magazine. With two magazines in docking compartment 1333, robot apparatus 150 through end effector 160 retrieves a handle from handle storage area 121 and places the handle on the adjacent magazines. FIG. 8 shows end effector 160 of robot apparatus 150 grasping handle 113 and placing handle 113 onto magazine 111a and magazine 111b in docking compartment 1333. Once handle 113 is in place, end effector 160 releases its grasp and robot arm 153 is retracted from loading retort 133. A magazine assembly of magazine 111a and magazine 111b is now ready for loading into a tissue processing module (e.g., pathology module 110). If pathology module 110 is ready, the magazine assembly of magazine 111a, magazine 111b and handle may be placed in a drip position above a fluid level in retort 133, such as in drip tray 1332 (e.g., end effector 160 of robot apparatus 150 grasps handle 113 and moves the magazine assembly to drip tray 1332).

Pathology module 110 such as a Tissue-Tek Xpress® may have an input door (input door 108, FIG. 2) engaged by an actuator that is engaged while the module is operating or otherwise cannot accept a magazine assembly and, when a magazine assembly can be accepted, the input door may be released by a signal from robot apparatus (e.g., a light signal sent from robot arm 153 to a sensor associated with the actuator). With a magazine assembly in loading retort 133 ready for processing, robot arm 153 may be positioned in proximity to an input door of pathology module 110 or a sensor associated with pathology module 110 to signal pathology module 110 for the opening of the input door (block 310, FIG. 15). When released by the actuator, the input door of pathology module 110 swings open to allow robot arm 153 access to the interior of the module.

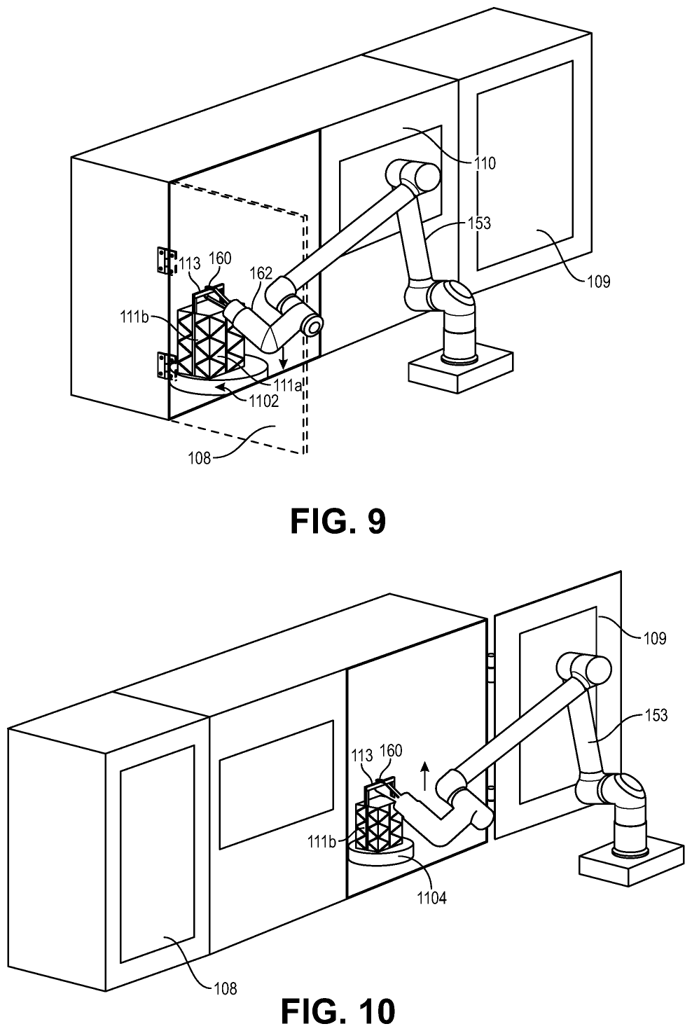

Once an input door of pathology module 110 is open, the control instructions associated with robot apparatus 150 direct robot arm 153 and end effector 160 to move to loading retort 133 in bench 130 and then grasp a magazine assembly in docking compartment 1333 by a handle. Once end effector has grasped a handle of a magazine assembly, the instructions direct that robot arm 153 remove the magazine assembly from loading retort 133 and transport the magazine assembly through input door 108 of processing module 110 (block 315, FIG. 15). FIG. 9 shows end effector 160 of robot arm grasping handle 113 and positioned at least partially through input door 108 within processing module 110. Once inside processing module 110, the instructions direct robot arm 153 to lower the magazine assembly into input retort 1102 in processing module 110 (block 320, FIG. 15). Input retort 1102 may, for example, be a cylindrical retort containing a pre-processing fluid. Following the lowering of the magazine into input retort 1102, the instructions direct end effector to release a grasp on the handle, remove robot arm 153 from within processing module 110 and to close input door 108 (block 325, FIG. 15). Input door 108 may be closed by robot arm 153 pushing the door closed from outside. Alternatively, the actuator associated with input door 108 may close the door electronically.

Once input door 108 of pathology module 110 is closed, processing module 110 may begin processing of tissue samples in each magazine (e.g., magazine 111a and magazine 111b) (block 330, FIG. 15). Tissue processing in pathology module 110 may begin automatically through sensors in processing module 110 that sense the closing of input door 108 or a magazine assembly in input retort 1102 (e.g., photo eye sensors associated with input retort 1102 may transmit a signal that a magazine assembly is presented).

In the example where pathology module 110 is a Tissue-Tek Xpress®, pathology module may perform tissue processing steps of, for example, dehydrating, clearing and impregnation of tissues in respective cassettes in magazine 111a and magazine 111b. Once the tissue processing steps are performed, a magazine assembly is placed in an output retort within pathology module 110. Robot apparatus 150 may periodically check pathology module 110 to detect the presence of a magazine assembly in an output retort of the module or a signal from pathology module 110 that a magazine assembly is present in the output retort. Alternatively, an output retort may include a sensor to alert robot apparatus 150 or computer 115 that a magazine assembly is present in the output retort. For example, an output retort may include photo eye sensors that detect the presence of a magazine assembly in the retort and transmit a signal to computer 115.

Once robot apparatus 150 identifies or is alerted to the presence of a magazine assembly in an output retort of pathology module 110, robot apparatus 150, through non-transitory, machine readable control sequences or instructions in robot controller 151 and possibly transmitted to robot controller 151 from computer 115, robot arm 153 may be positioned in proximity to output door 109 of pathology module 110 or a sensor associated with pathology module 110 to signal pathology module 110 for the opening of the output door (block 35, FIG. 15). When released by the actuator, output door 109 of pathology module 110 swings open to allow robot arm 153 access to the interior of the module. Control instructions associated with robot apparatus 150 then direct robot arm 153 to move through output door 109 into pathology module 110 and grasp a handle of a magazine assembly and remove the magazine (block 340, FIG. 15). FIG. 10 shows end effector 160 grasping handle 113 of a magazine assembly including magazine 111a and 111b and lifting the magazine assembly from output retort 1104 in pathology module 110.

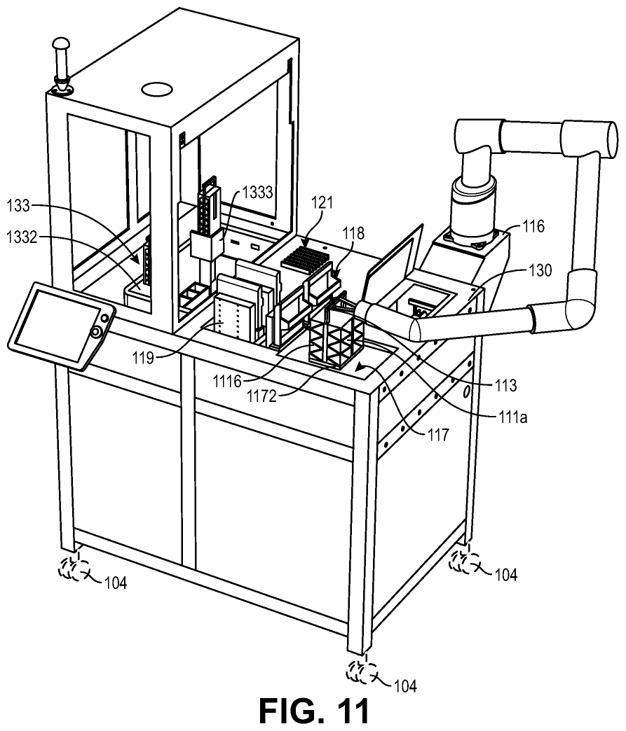

Upon removing a magazine assembly from output retort 116, the instructions to control robot arm 153 may direct the robot arm to place each magazine of the magazine assembly (magazine 111a, magazine 111b) in pathology module 120. Pathology module 120 may be a module that performs paraffin embedding of tissue cassettes. Where pathology module 120 is a Tissue-Tek AutoTEC®, the embedding module is designed to accommodate the introduction of magazines individually, not as a magazine assembly of two magazines. Accordingly, in such instance, a handle on a magazine assembly needs to be removed and the magazines separated. In such instances, the instructions to control arm 153 upon removal of a magazine assembly from output retort 116 of pathology module 110 may direct the transport of a magazine assembly to bench 130 and removal of the handle by end effector 160. At bench 130, the magazine assembly may be placed in a slot or compartment in magazine storage area 116. The slot or compartment in magazine storage area 116 provides the magazine assembly with support to maintain a stationary position during removal of a handle of the magazine. FIG. 11 shows magazine assembly including magazine 111a and magazine 111b and handle 113 in compartment 1172 of magazine storage area 117. FIG. 11 also shows end effector 160 of robot apparatus 150 grasping handle 113 of the magazine assembly and pushing the handle in a direction toward the rear of each magazine to separate handle 113 from each magazine.

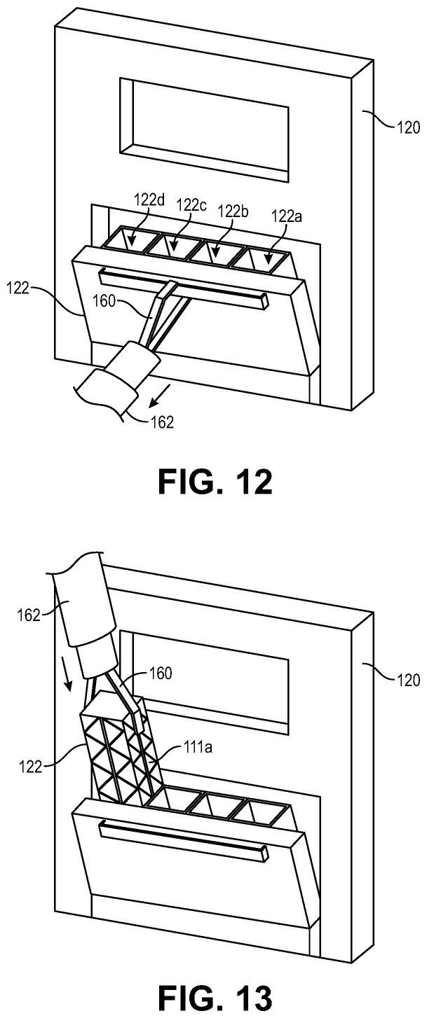

Upon separating the handle from a magazine assembly, non-transitory, machine readable control sequences or instructions in robot controller 151 and possibly transmitted to robot controller 151 from computer 115 may direct end effector into a position to grasp a handle on an input door of pathology module 120 and open the door (block 345, FIG. 15). FIG. 12 shows a view of end effector 160 grasping a handle of input door 122 of pathology module 120 and opening the door. An inside of input door 122 of a Tissue-Tek AutoTEC® includes a number of slots into respective ones of which can be placed individual magazines. FIG. 12 shows slot 122a, slot 122b, slot 122c and slot 122d.

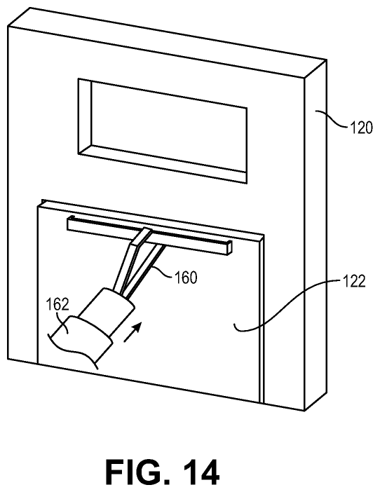

Once input door 122 is opened by robot arm 153, the instructions may direct end effector 160 to release the grasp on the door handle and to return to bench 130. At bench 130, robot arm 153 may be directed by instructions to grasp an individual magazine from magazine storage area (e.g., magazine 111a), transport the grasped magazine to a position over input door 122 and to load the magazine into a slot in the door (block 350 and block 355, FIG. 15). FIG. 13 shows end effector 160 grasping a top end of magazine 111a and lowering the magazine into slot 122d in input door 122 of pathology module 120. Once the magazine is lowered into a slot, the instructions may direct end effector 160 to release its grasp and robot arm 153 to return to bench 130 and transport to and load one or more other magazines (e.g., magazine 111b) in pathology module 120. Once the magazine loading is complete, the instructions may direct end effector 160 to grasp the handle of input door 122 and close the door (block 360, FIG. 15). FIG. 14 shows end effector 160 grasping the handle and robot arm closing input door 122.

When one or more magazines are placed in input door 122 and the door is closed, pathology module 120 of a Tissue-Tek AutoTEC® may begin embedding of tissue samples in each magazine (block 365, FIG. 15). Such processing may begin automatically through sensors in processing module 120 (e.g., photo eye sensors associated with each of slot 122a-122d may transmit a signal that a magazine is presented and door 122 is closed).

Where pathology module 120 is a Tissue-Tek AutoTEC®, embedding of individual tissue cassettes begins by removing the tissue cassettes from a respective magazine. Tissue cassettes are then individually processed to embed the tissue in paraffin. After embedding, a tissue cassette is automatically placed in a slot of an output door. FIG. 2 shows pathology module 120 having output door 126a, output door 126b, output door 126c and output door 126d attached to the module. Output door 126e is shown in storage area 118 in surface portion 131b of bench.

When processing (e.g., tissue embedding) in pathology module 120 is complete, pathology module 120 may include a sensor to alert robot apparatus 150 or computer 115 that processing therein is finished and, in the case of a Tissue-Tek AutoTEC®, that one or more cassettes are in an output door (output door 126a-126d).

Once robot apparatus 150 is alerted that processing in pathology module 120 is complete, robot apparatus 150, through non-transitory, machine readable control sequences or instructions in robot controller 151 and possibly transmitted to robot controller 151 from computer 115 may direct end effector 160 at a distal end of robot arm 153 to move into a position to grasp a handle on an output door of the module. In the example where pathology module 120 is a Tissue-Tek AutoTEC®, the instructions may direct end effector 160 into a position to grasp a handle of one of output door 126a-126d and remove the door from the pathology module (block 370 and block 375, FIG. 15). Inset B in FIG. 2 representatively shows end effector 160 grasping output door 126e free of the pathology module. The instructions may direct robot arm to transfer the output door to bench 130 or other area and end effector 160 to then release a grasp on the output door.

Where pathology module 120 is a Tissue-Tek AutoTEC®, as noted above, tissue cassettes are removed from magazines within the module. The empty magazines may be left within input door 122 of pathology module 120. Such empty magazines may be removed robot apparatus 150. For example, control sequences or instructions in robot controller 151 may direct robot arm 153 to return to input door 122 after robot arm has loaded one or more magazines in the door and closed the door. The instructions may direct end effector 160 to grasp a handle of input door 122 of pathology module 120 and open the door to expose an inside of input door 122. The instructions may also direct end effector to release a grasp on the handle and for robot arm 153 to move into a position over a slot in the door (e.g., slot 122a-122d, FIG. 12) and to grasp any magazine in the slot The instructions may further direct that the magazine (an empty magazine) be removed from the slot and transferred to bench 130 or some other area. The process of positioning robot arm 153 over a slot in the door, grasp a magazine in the slot, remove the magazine and transfer the magazine may be repeated until all the empty magazines are removed from pathology module 120. At that point, the instructions may further direct that robot arm 153 close input door 122.

The above description described the incorporation and use of a robotic assembly to transfer tissue cassettes between pathology modules in a pathology or histology laboratory frees up valuable time and energy of highly skilled operators and may offer more reliable and efficient throughput of tissue samples in the laboratory as idle times where an operator is not available to unload or load a pathology module may be reduced. The incorporation and use also will tend to reduce exposure of operators (lab personnel) to noxious fumes from reagents such as alcohols, fixatives and others associated with one or more pathology modules. The tracking functionality associated with the reading/capturing and recording/comparing in computer 115 may provide tracking or tracing of individual cassettes throughout a laboratory process, minimize human errors and serve the additional benefit of monitoring reagent usage and supply levels in pathology modules. For example, by recording the cassette identifier information, computer 115 may also track the use of reagents in a pathology module (e.g., the number of cassettes through versus an amount of a reagent). This allows supply and inventory levels of reagents to be controlled.

The incorporation and use of a robotic assembly to transfer tissue cassettes between pathology modules in a pathology or histology laboratory also provides flexibility in allowing pathology modules to be used independently enabling prioritization of histological cases and drop off and pick up of histological cases at a discretion of an operator.

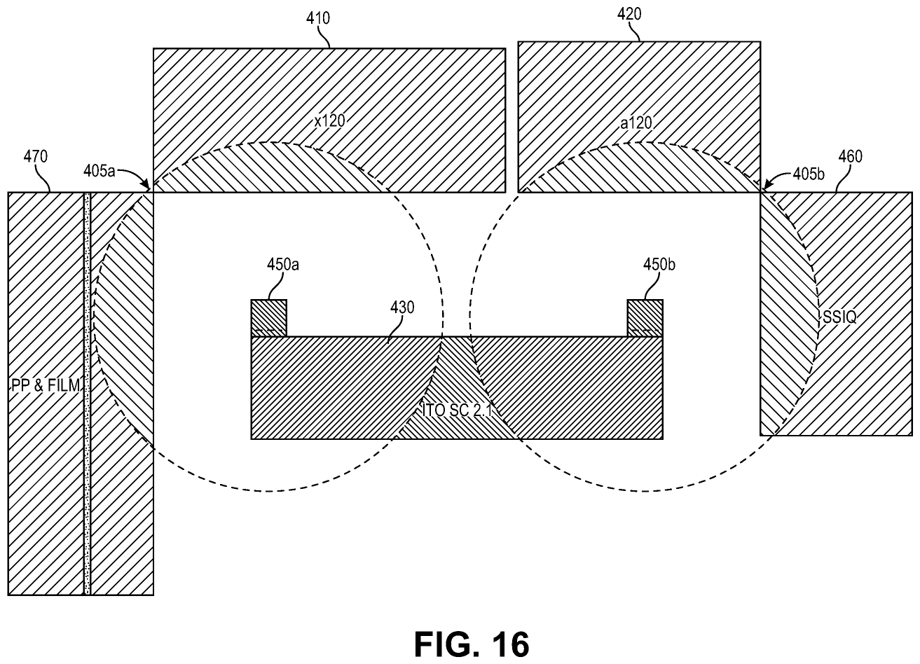

While the above portion of a pathology or histology laboratory included two pathology modules (a tissue processing instrument and a tissue embedding instrument), it is appreciated that other pathology modules may be substituted or included in an accessible region for a robotic assembly, such as accessible region 105, including but not limited to sectioning stations, microtomy stations, staining instruments, coverslipping instruments and imaging instruments. It is also appreciated that more than one robotic assembly may be present in an accessible region or that a robotic assembly may include more than one robotic arm. FIG. 16 shows a top view of a portion of a pathology or histology laboratory including four pathology modules and two robotic transfer assemblies associated with a bench and indicates an accessible region in which each robotic transfer assembly can function. In this example, pathology module 410 may be a tissue processing module such as a Tissue-Tek Xpress®, pathology module 420 may be an embedding module such as a Tissue-Tek AutoTEC®, pathology module 460 may be a grossing station or microtomy module operable to produce slides having sections of tissue samples thereon, and pathology module 470 is a staining module operable to stain tissue samples on slides or combined staining and coverslipper module. One example is a Tissue Tek Prisma® Plus and Tissue-Tek Film® coverslipper. FIG. 16 also shows first robotic assembly or apparatus 450a and second robotic assembly or apparatus 450b each positioned at opposite ends of bench 430 and representative accessible region 405a for first robotic assembly 450a and accessible region 405b for second robotic assembly 450b. Accessible regions for particular robotic assemblies may be adjacent one another or overlapping so that a robotic assembly in one accessible region may interact with a robotic assembly or carrier in another accessible region of a laboratory. A base of one or both of first robotic assembly 450a and second robotic assembly 450b may be connected to bench 430 at a fixed location and may be operable to move along a side of bench 430 such as on a rail connected to the bench. It is appreciated that one robotic assembly may be employed to service each of the modules. Bench 430 may be similar to bench 130 including similar areas for storage or processing (e.g., identifier capturer/reader). Bench 430 may be movable, such as a motorized bench on wheels or rollers that is electrically movable through a portion of a pathology or histology laboratory. Alternatively, at least one of first robotic assembly 450a and second robotic assembly 450b are a free-standing assembly positioned in this illustration adjacent bench 430. At least one of first robotic assembly 450a and second robotic assembly 450b as a free-standing unit may include a stand that includes wheels or rollers and a motor (e.g., an electric motor) that can be controlled by a computer (e.g., computer 115) to maneuver the at least robotic assembly through accessible region 405a and/or accessible region 405b. As another alternative, one or both of first robotic assembly 450a and second may be connected to a wall, a ceiling or a gauntry.

In still other examples, accessible regions for particular robotic assemblies may be adjacent one another or overlapping so that a robotic assembly in one accessible region may interact with a robotic assembly or carrier in another accessible region of a laboratory.

The following numbered clauses summarize some aspects of the invention:

1. A method comprising:
engaging a carrier with a robot arm; and
transferring the carrier by the robot arm from a first location outside a pathology instrument to a second location in the pathology instrument.

2. The method of clause 1, wherein the carrier is operable to contain a plurality of tissue cassettes.

3. The method of clause 1 or clause 2, wherein the carrier is a magazine and the magazine comprises a handle and the robot arm comprises an end effector, wherein transferring the carrier by the robot comprises gripping the handle by the end effector.

4. The method of any of the preceding clauses, wherein the pathology instrument is a tissue processing instrument.

5. The method of clause 4, further comprising removing the carrier by the robot arm from the tissue processing instrument.

6. The method of any of clause 1 through clause 4, wherein the pathology instrument is an embedding instrument and the carrier is a first carrier comprising at least one tissue cassette and the method further comprises removing a second carrier comprising the at least one tissue cassette by the robot arm.

7. The method of any of the preceding clauses, wherein the pathology instrument is first pathology instrument and the method further comprises:
transferring the carrier by the robot arm from the first pathology instrument to a second pathology instrument.

8. The method of any of clause 1 through clause 5, wherein the first location is a grossing station.

9. A pathology assembly comprising:
a first pathology module;
a second pathology module; and
a robot arm operable to transfer a carrier operable to contain a plurality of tissue cassettes between the first pathology module and the second pathology module.

10. The pathology assembly of clause 9, wherein the first pathology module comprises a grossing station.

11. The pathology assembly of clause 9 or clause 10, wherein the second pathology module comprises a tissue processing instrument.

12. The pathology assembly of clause 11, wherein the first pathology module comprises a grossing station and the assembly further comprises a third pathology module wherein the robot arm is further operable to transfer the carrier from second pathology module to the third pathology module.

13. The pathology assembly of clause 12, wherein the carrier is a first carrier and the robot arm is further operable to remove a second carrier from the third pathology module.

14. The pathology assembly of clause 12, wherein the third pathology module comprises an embedding instrument.

15. The pathology assembly of any of clause 9 through clause 14, further comprising a reader or capturer operable to read or capture an identifier on the carrier.

16. The pathology assembly of clause 15, wherein the reader or capturer is a bar code reader.

17. A pathology assembly comprising:
a robot arm coupled to a base; and
a processor coupled to the robot arm and comprising non-transitory machine readable instructions that when executed cause the robot arm to perform a method comprising:
   engaging a carrier with the robot arm; and
   transferring the carrier by the robot arm from a first location outside a pathology instrument to a second location in the pathology instrument.

18. The pathology assembly of clause 17, further comprising a reader or capturer operable to read or capture an identifier on the carrier.

19. The pathology assembly of clause 17 or clause 18, wherein the method further comprises transferring the carrier by the robot arm from the first pathology instrument to a second pathology instrument.

20. The pathology assembly of any of clause 17 through clause 19, wherein the base of the robot arm is operable to be moved from a first position to a second position.

21. The pathology assembly of any of clause 17 through clause 19, wherein the base is coupled to a bench or moveable cart and wherein the bench or moveable cart comprises the reader or capturer and optionally one or more storage areas, or wherein the base is free-standing and moveable about an accessible region including the first location and the second location or wherein the base is coupled to a ceiling, wall or gantry.

In the description above, for the purposes of explanation, numerous specific details have been set forth in order to provide a thorough understanding of the embodiments. It will be apparent however, to one skilled in the art, that one or more other embodiments may be practiced without some of these specific details. The particular examples described are not provided to limit the invention but to illustrate it. The scope of the invention is not to be determined by the specific examples provided above but only by the claims below. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that in the description various features are sometimes grouped together in a single example, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. A method comprising:
engaging a carrier with a robot arm; and
transferring the carrier by the robot arm from a first location outside a pathology instrument to a second location in the pathology instrument.

2. The method of claim 1, wherein the carrier is operable to contain a plurality of tissue cassettes.

3. The method of claim 1, wherein the carrier is a magazine and the magazine comprises a handle and the robot arm comprises an end effector, wherein transferring the carrier by the robot comprises gripping the handle by the end effector.

4. The method of claim 1, wherein the pathology instrument is a tissue processing instrument.

5. The method of claim 4, further comprising removing the carrier by the robot arm from the tissue processing instrument.

6. The method of claim 1, wherein the pathology instrument is an embedding instrument and the carrier is a first carrier comprising at least one tissue cassette and the method further comprises removing a second carrier comprising the at least one tissue cassette by the robot arm.

7. The method of claim 1, wherein the pathology instrument is first pathology instrument and the method further comprises:
transferring the carrier by the robot arm from the first pathology instrument to a second pathology instrument.

8. The method of claim 1, wherein the first location is a grossing station.

9. A pathology assembly comprising:
a first pathology module;
a second pathology module; and
a robot arm operable to transfer a carrier operable to contain a plurality of tissue cassettes between the first pathology module and the second pathology module.

10. The pathology assembly of claim 9, wherein the first pathology module comprises a grossing station.

11. The pathology assembly of claim 9, wherein the second pathology module comprises a tissue processing instrument.

12. The pathology assembly of claim 11, wherein the first pathology module comprises a grossing station and the assembly further comprises a third pathology module wherein the robot arm is further operable to transfer the carrier from second pathology module to the third pathology module.

13. The pathology assembly of claim 12, wherein the carrier is a first carrier and the robot arm is further operable to remove a second carrier from the third pathology module.

14. The pathology assembly of claim 12, wherein the third pathology module comprises an embedding instrument.

15. The pathology assembly of claim 9, further comprising a reader or capturer operable to read or capture an identifier on the carrier.

16. The pathology assembly of claim 15, wherein the reader or capturer is a bar code reader.

17. A pathology assembly comprising:
a robot arm coupled to a base; and
a processor coupled to the robot arm and comprising non-transitory machine readable instructions that when executed cause the robot arm to perform a method comprising:
engaging a carrier with the robot arm; and
transferring the carrier by the robot arm from a first location outside a pathology instrument to a second location in the pathology instrument.

18. The pathology assembly of claim 17 further comprising a reader or capturer operable to read or capture an identifier on the carrier.

19. The pathology assembly of claim 17, wherein the method further comprises transferring the carrier by the robot arm from the first pathology instrument to a second pathology instrument.

20. The pathology assembly of claim 17, wherein the base of the robot arm is operable to be moved from a first position to a second position.
